(19) United States
(12) Patent Application Publication
(10) Pub. No.: US 2006/0292107 A1
(43) Pub. Date: Dec. 28, 2006

(54) Solid shaving composition and packaging system

(76) Inventor: Benjamin W. McCormick, Charlotte, NC (US)

Correspondence Address: DOUGHERTY CLEMENTS, 1901 ROXBOROUGH ROAD, SUITE 300, CHARLOTTE, NC, 28211, US

(21) Appl. No.: 11/166,014

(22) Filed: Jun. 24, 2005

(51) Int. Cl. A61K 8/73

(52) U.S. Cl. 424/73

(57) ABSTRACT

A method of packaging and distributing substantially dehydrated shaving strips, wherein a solid strip of substantially dehydrated shaving lotion is provided. The shaving strips are cut or pressed into discreet units. Alternatively, the dehydrated shaving composition can be deposed onto a water soluble carrier strip. A plurality of shaving strips are then packaged into a container and sold as a unit. A user can then remove one or more of the shaving strips from the container, and mix with water to hydrate the shaving cream.

### FIELD OF THE INVENTION

The present invention relates to a methods of packaging and compositions for solid shaving strips, and more particularly to a method of packaging shaving compositions that have been compressed or dehydrated into thin strips, and stacked I small container for distribution and use.

### BACKGROUND OF THE INVENTION

Shaving creams, foams, and gels are most commonly available contained within a large canister, along with a propellant gas, or some other dispensing method for ejection of the shaving cream. These cans are usually of sizes that make them impracticable for travel. Furthermore, in a depressurized cargo area of many airplanes, these canisters will often leak large amounts of the shaving composition into the traveler's luggage, which can damage the traveler's other belongings.

These shaving creams generally contain water, a soap, and a propellant gas. Additionally, shaving creams may contain foam builders, stabilizers, wetting agents, emollients, cleansing agents, humectants, and other compositions that may impart desirable properties to the creams. In post-foaming compositions, a post-foaming agent, such as a hydrocarbon, is also present in the composition that causes the gels to foam upon application to the user's face.

The large shaving canisters used today are not only an inconvenience to the consumer, but also to the retailer and manufacturer. The weight, size and composition of the canister contribute to higher freight costs, considerable use of valuable retail shelf space, and environmental concerns in recycling.

While there have been numerous improvements to the cream compositions, the basic method of packaging these creams has remained unchanged. Creams are still distributed in the large, unwieldy canisters.

Therefore, there is a need in the art for a shaving composition that is easily portable, has a smaller footprint in a retail environment, and will not leak from its container in travel situations.

### SUMMARY OF THE INVENTION

In accordance with the present invention, there is provided a solid, rapidly dissolvable, and readily foamable shaving composition. The composition is substantially free of water, and is compressed into thin sheets. The thin sheets can themselves be cut into strips for single use dosages, which can be individually packaged or stacked into groups, and placed into a small tin.

The strips are comprised substantially of a soap base, but may also include emollients, fragrances, colorants, foaming agents, humectants, thickeners, and agents to adjust the lubricating properties of the resulting foam.

When compressed into thin sheets, the shaving composition may additionally contain a binder to retain the composition in sheet form.

In another embodiment of the invention, an improved method for packaging shaving strips is provided. Shaving compositions according to the current invention can be condensed or compressed into thin sheets, which are then cut into small strips for single uses. A plurality of the strips can then be stacked atop each other, and placed within a small tin.

In another embodiment of the invention, an improved method of shaving is provided. A substantially solid, compressed or condensed shaving strip is removed from a retaining tin. The shaving strip is then placed in the user's hands, with a small amount of water. The user rubs his hands together, causing the shaving strip to dissolve in the amount of water, and create a thick, viscous foam suitable for shaving a person's face, legs, or other body parts.

### OBJECTS OF THE INVENTION

It is a principle object of the invention to provide a new method of packaging and manufacturing a shaving composition.

It is another object of the invention to provide a new method of packaging a shaving composition wherein the shaving composition is substantially free of water, and has been formed into thin shaving strips.

It is another object of the invention to provide a new method of packaging a shaving composition wherein the shaving composition is substantially free of water, and has been deposited onto a water soluble carrier strip.

It is another object of the invention to provide a new method of packaging a shaving composition wherein a plurality of shaving strips are provided and are stacked on top of one another, so that a plurality of shaving strips can be packaged into a small tin.

It is another object of the invention to provide a new method of shaving wherein a shaving strip is agitated with an effective amount of water, creating a viscous foam that can then be applied to unwanted body hair, the body hair then being removed with a razor.

It is another object of the current invention to provide a new shaving composition, comprising a soap base, and less than 5% water, that can be readily formed into thin strips and deposited onto water soluble carrier strips.

### BRIEF DESCRIPTION OF THE DRAWING

FIG. 1 is a perspective view of the invented shaving strip, showing a number of different non-limiting strip designs.

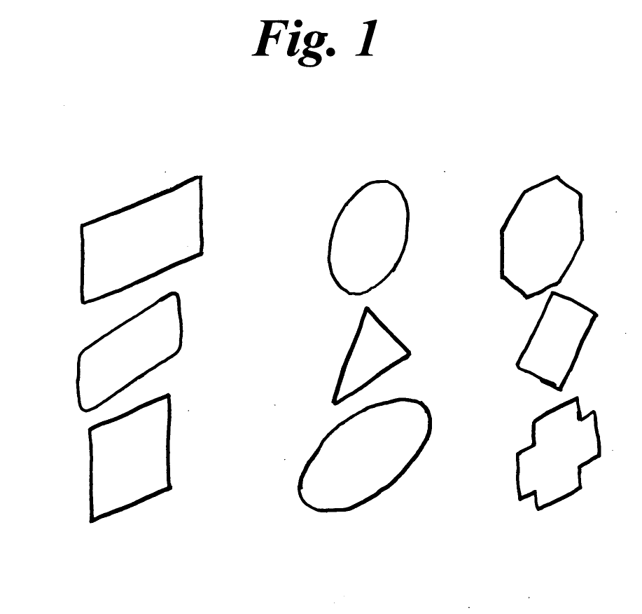

### DETAILED DESCRIPTION

The shaving composition of the present invention is in the compressed form of a substantially solid strip. The strip itself can be extruded, pressed, poured and cast, or otherwise formed such that it is less than about 0.5 mm in thickness, and preferably less than 0.1 mm. The strip contains water, a solid soap composition, and optionally can contain thickeners, fatty acids, humectants, emollients, antioxidants, perfumes, and coloring agents.

The shaving composition contains only an effective amount of water necessary to retain stability of the strips, and to maintain a homogenous composition in the strip. The amount of water is not so great as to cause excess flow, such that a plurality of strips placed in close proximity should not fuse together. Preferably, an effective amount of water is less than 5% by weight.

A variety of solid soaps, as are known in the art, may be employed, such as sodium and/or potassium cocoate, stearate, palmitate, myristate and mixtures thereof. In addition to providing the main lather-building ingredient of the composition, the soap also can impart some deodorizing and antimicrobial activity to the composition as well. The soap composition usually comprises from about 70 to about 95 wt. % of the shaving composition.

The shaving compositions of the present invention can also include a water soluble thickening agent suitable for thickening the composition to a lotion form upon addition of water to the shaving strip. Thickeners are effective in the composition in amounts greater than 0.1 wt. %, but can be present in amounts up to about 20 wt. %. Preferably, the thickener is a glycol, but can also be other components that are known in the art. Suitable water soluble thickening agents for inclusion in the shaving strip generally include alkyl glycols, polyalkyl glycol ethers, polyacrylic acids, polyacrylamides, modified cellulose polymers, gums, resins, and starches.

The optional humectant component is added as a skin moisturizing agent in amounts up to 20 wt %. While glycerin is most widely utilized, other suitable alternatives include propylene glycol, sorbitol, hydrogenated corn syrup, sodium pyrrolidone carboxylate and mixtures thereof.

Optionally, a self-foaming agent can be included in the shaving composition to aid in foam formation. The self-foaming agent may be any volatile hydrocarbon or halohydrocarbon with a sufficiently low boiling point that it will volatilize and foam the lotion upon application to the skin, but not so low that it causes the lotion to foam prematurely. The typical boiling point of such an agent falls within the range of −20 degrees to 40 degrees C. Unlike shaving creams and gels of the prior art, the invented shaving composition is intended to be stored at ambient temperature and pressure, and therefore any included self-foaming agents should have a boiling point greater than room temperature.

Other compatible additives known in the art for use in shaving preparations may also be included in minor proportions, so long as they do not adversely affect the properties of the shaving strip. Such additives generally include emollients, antiseptics, skin soothers, aloe, lanolin, menthol, stability enhancers, coloring agents, and perfumes.

The composition can be pressed, poured and cast, rolled, or extruded into sheets. Preferably, the shaving composition is cast on a releasable carrier film and dried. The carrier film is made of a material that allows the shaving solution to spread evenly across the carrier film width without the occurrence of bonding between the shaving composition and carrier. Drying of the carrier film may be carried out at high temperature using a drying oven, drying terminal, vacuum drier, or any other suitable drying equipment, which does not adversely affect the ingredients of which the carrier film is composed. Drying conditions should not be such that would cause any included self-foaming agent to volatize.

Optionally, the shaving composition can be cast onto a water soluble carrier film. Acceptable films include, but are not limited to, acetate type films, such as polyvinyl acetate polymer film, and cellulose type films, such as a low viscosity hydropropylmethyl cellulose polymer.

The film, once formed, is segmented into single dosage units, or strips, by die-cutting or slitting-and-die-cutting. The single dosage strip is generally small, so that a single unit produces enough shaving lather upon hydration for a single shave. The size of the dosage strip can be varied to provide enough lather to shave a larger or smaller area, i.e., a face or legs.

After cutting the sheet into single dose strips, the shaving strips may be stacked atop each other and a plurality of strips packaged into a small container. In order to prevent adherence of the strips to one another, the strips can be coated with silicone or other acceptable dissolvable coating material that does not interfere with the foaming action of the shaving composition, nor irritate the user's skin.

The container itself is substantially smaller than shaving cream canisters that are known in the prior art. Ideally, the invented container tin is just large enough to accommodate a stack of the invented shaving strips. The preferred embodiment provides a container tin that allows for a single stack of the invented shaving strips. However, larger container tins can be utilized, providing more than one stack of the invented shaving strips.

In use, a user removes a single shaving strip from the container. The user of the strip then mixes the shaving strip with an effective amount of water to hydrate the shaving composition, and works the strip into a stiff lather. The lather can then be applied to the user's face, legs, or other body part. The user can then shave with a regular wet or dry razor.

### SUMMARY OF THE ACHIEVEMENT OF THE OBJECTS OF THE INVENTION

From the foregoing, it is readily apparent that I have invented an improved method of packaging and manufacturing a shaving composition, and a composition for such packaging.

Further, it is readily apparent that I have invented an improved method of packaging a shaving composition wherein the shaving composition is substantially free of water, and has been formed into thin shaving strips.

Further, it is readily apparent that I have invented an improved method of packaging a shaving composition wherein the shaving composition is substantially free of water, and has been deposited onto a water soluble carrier strip.

Further, it is readily apparent that I have invented an improved method of packaging a shaving composition wherein a plurality of shaving strips are provided and are stacked on top of one another, so that a plurality of shaving strips can be packaged into a small tin.

Further, it is readily apparent that I have invented an improved method of shaving wherein a shaving strip is agitated with an effective amount of water, creating a viscous foam that can then be applied to unwanted body hair, the body hair then being removed with a razor.

Further, it is readily apparent that I have invented an improved shaving composition, comprising a soap base, and less than 5% water, that can be readily formed into thin strips and deposited onto water soluble carrier strips.

It is to be understood that the foregoing description and specific embodiments are merely illustrative of the best mode of the invention and the principles thereof, and that various modifications and additions may be made to the apparatus by those skilled in the art, without departing from the spirit and scope of this invention, which is therefore understood to be limited only by the scope of the appended claims.

I claim:

1. A method for shaving, said method comprising:
providing a plurality of substantially dehydrated shaving strips;
selecting at least one of said plurality of shaving strips;
adding an effective amount of water to said shaving strip and agitating said amount of water with said shaving strip to form a viscous lather;
applying said lather to a body part; and
shaving said body part with a razor.

2. The method of claim 1, wherein said shaving strips are disposed onto a water soluble carrier film.

3. The method of claim 2, wherein said carrier film is selected from the group consisting of acetate films and cellulose films, or a combination thereof.

4. The method of claim 3, wherein said acetate film is a polyvinyl acetate polymer film.

5. The method of claim 3, wherein said cellulose film is a low viscosity hydropropylmethyl cellulose polymer.

6. A method of packaging a shaving composition, said method comprising:
providing a plurality of substantially dehydrated shaving strips;
stacking said plurality of shaving strips in an orderly arrangement atop each other; and
packaging said stacked plurality shaving strips within a container tin.

7. The method of claim 6, further comprising the steps of:
providing a water soluble carrier film, and
deposing said shaving strips onto said carrier film.

8. The method of claim 7, wherein said carrier films are selected from the group consisting of acetate films and cellulose films, or a combination thereof.

9. The method of claim 8, wherein said acetate film is a polyvinyl acetate polymer film.

10. The method of claim 8, wherein said cellulose film is a low viscosity hydropropylmethyl cellulose polymer.

11. A substantially dehydrated shaving strip having a thickness of less than 0.5 mm, said shaving strip comprising:
from about 70 to about 95 wt. % solid soap composition;
from about 0.1 to about 5 wt. % water;

12. The shaving composition of claim 11, wherein said soap composition is selected from the group consisting of sodium cocoate, potassium cocoate, stearate, palmitate, myristate and mixtures thereof

13. The shaving strip of claim 11, further comprising from about 0.1 to about 20 wt. % thickener.

14. The shaving strip of claim 13, wherein said thickener is selected from the group consisting of alkyl glycols, polyalkyl glycol ethers, polyacrylic acids, polyacrylamides, modified cellulose polymers, gums, resins, and starches.

14. The shaving strip of claim 11, further comprising from about 0.1 to about 20 wt. % humectants.

15. The shaving strip of claim 14, wherein said humectants are selected from the group consisting of glycerin, propylene glycol, sorbitol, hydrogenated corn syrup, sodium pyrrolidone carboxylate and mixtures thereof.

16. The shaving strip of claim 11, further comprising a self-foaming agent.

17. The shaving strip of claim 11, further comprising emollients, antiseptics, skin soothers, aloe, lanolin, menthol, stability enhancers, coloring agents, perfumes, or combinations thereof.

18. The shaving strip of claim 11, further comprising a water soluble carrier film on which the shaving strip is adhered or disposed.

19. The shaving strip of claim 18, wherein said carrier films are selected from the group consisting of acetate films and cellulose films, or a combination thereof.
